(19) United States
(12) Patent Application Publication
(10) Pub. No.: US 2003/0154035 A1
(43) Pub. Date: Aug. 14, 2003

(54) Simulation of gene expression control using connectrons, interference RNAs (iRNAs) and small temporal RNAs (stRNAs) in prokaryotic, archea and eukaryotic genomes

(76) Inventor: Richard J. Feldmann, Derwood, MD (US)

Correspondence Address: Law Office of Jim Zegeer, Suite 108, 801 North Pitt Street, Alexandria, VA, 22314, US

(21) Appl. No.: 10/339,666

(22) Filed: Jan. 10, 2003

Related U.S. Application Data
(63) Continuation-in-part of application No. 10/227,568, filed on Aug. 26, 2002, now abandoned.
Application No. 10/227,568 is a continuation-in-part of application No. 09/866,925, filed on May 30, 2001.
(60) Provisional application No. 60/347,295, filed on Jan. 14, 2002.

(51) Int. Cl. G06F 19/00; G01N 33/48; G01N 33/50

(52) U.S. Cl. 702/20

(57) ABSTRACT

A computer method for the determination of the interaction between transient and permanent connectrons, interference RNA and small temporal RNA.

### REFERENCE TO RELATED APPLICATION

The present application is the subject of Provisional Application Serial No. 60/347,295 filed Jan. 14, 2002

The present application is a continuation in part of U.S. patent application Ser. No. 09/866,925 filed May 30, 2001 entitled ALGORITHMIC DETERMINATION OF FLANKING DNA SEQUENCES THAT CONTROL THE EXPRESSION OF SETS OF GENES IN PROKARYOTIC, ARCHEA AND EUKARYOTIC GENOMES, incorporated herein by reference.

The present application is an continuation in part of U.S. patent application Ser. No. 10/227,568 filed Aug. 26, 2002 entitled Determination of flanking DNA sequences that control the expression of sets of genes in the Escherichia coli K-12 MG1655 complete genome, incorporated herein by reference.

### INTRODUCTION

The connectron structure of a genome determines sets of four DNA sequences (called C1, C2, T1 and T2) of minimum length of 15-bases (C1 and C2 which are in the 3′UTR of a gene, T1 which is on the 5′-side and T2 which is on the 3′-side of a set of genes). Typical genomes have from hundreds to tens of thousands of these tetradic relationships spread throughout the genome. When a gene is transcribed into RNA the C1 and C2 sequences in the 3′UTR find the cognate T1 and T2 double-stranded DNA sequences to form a pair of triple-stranded RNA-DNA-DNA generalized Hoogsteen helices. The genes between T1 and T2 are condensed into 30 nm chromatin structure and they are no longer open to promotion and transcription. The lifetime of each connectron is proportional to the length of the shorter of the two generalized Hoogsteen helices. Within a set of genes that have been removed from promotability by the formation of a connectron there may be genes that themselves have the same or different C1/C2 sequences in their 3′UTRs. This inclusion process induces a temporal dynamic because genes that are included in a connectron can no longer produce the source C1/C2 RNA sequences to form other connectrons. One of the most obvious instances of this temporal dynamic are the so-called “one-shot” connectrons in which the transcription of a gene produces a C1/C2 sequence pair that forms a connectron that includes the transcribed gene itself thus turning off the further expression of the gene. In general, however, the connectron sources (i.e. the C1/C2 sequences) and the connectron flanking targets (i.e. T1 and the T2 sequences) are in different portions of the genome. The evolutionary configuration of each genome alone determines whether the genes turned off by one connectron are associated with other connectrons.

The C1/C2 sequences that are the sources of connectrons can also bind to the DNA double-stranded sequences of other equivalent C1/C2 sequences in the 3′UTR of other genes. Where these trip-stranded RNA-DNA-DNA generalized Hoogsteen helices form, the translation of the DNA into RNA is halted and no additional C1/C2 connectron source sequences are produced. This interference RNA (iRNA) produces an additional temporal dynamic. Once again the lifetime of this iRNA is varies directly with the length of the C1 and C2 sequences. Only the relative lengths of the lifetimes distinguish iRNAs from small temporal RNAs (stRNAs). The iRNA and stRNA modulate the temporal behavior of the connectrons.

The third type sequence-determined component that produces a temporal dynamic is the permanent connectron. If all the C1/C2 sources of a given connectron can be turned off by the action of other connectrons, then it is called a “transient connectron”. If, however, the generation of the C1/C2 source of a connectron is controlled only by promotion of its associated gene then the connectron is described as being “permanent”. The gene and its 3′UTR are always open to transcription and hence the C1/C2 RNA could be continually produced. Permanent connectrons have a dominant role in the temporal dynamic. Since the permanent connectrons cannot be altered by any subsequent connectron, RNAi or stRNA events, they act to determine in large measure temporal activity of the whole cell. As the documentation of genes for many of the different genomes publically available on the National Center for Biomedical Information (NCBI) server improves, the number of permanent connectrons detected by our basic-methods algorithm is becoming fewer and fewer.

An analogy will help to make the roles of the three sequence-determined components clearer. A musical organ is a device with three control components and one notation component. There are the stops that act to connect the tone-producing pipes to the keys on different keyboards. The pedals act to modulate the tones produced by individual key actions at a given time. In an organ the keys on different keyboards are depressed in a variety of sequences to produce the melody. The pedals are depressed in a somewhat slower fashion to produce different harmonies. As a composition moves from one phase to another, the organist will often change the pattern of the stops. The tempo of the composition is mainly determined by the rapid alteration of key depressions on the different keyboards. Unlike a piano or a harpsichord where such an action produces little effect, in organ music a given key can sometimes be held down for a relatively long time. In the same way, a pedal can be depressed for just a short time to produce just the hint of a harmony. The dynamic range of organ music, especially in an ancient cathedral, produces a sense of awe in most minds. The temporal behavior of cell is really very similar and just as full of awe. The connectrons interact with each other to produce most of the rapid changes in gene expression. Sets of genes (where a set can be one gene or many genes) are turned-off and, when the lifetime of the connectron expires, turned-on again. Since the lifetime of a connectron is determined by the length of the minimum intersecting sequences, some connectron lifetimes are very short while others are quite long. The iRNAs and the stRNA produced by gene expression also have lifetimes so they too can act in short-term or long-term fashions. In the same way that the pedals act to modulate the effect of the keys, the iRNAs and stRNAs act to modulate the temporal behavior and interaction of the connectrons. The different keyboards in an organ correspond to the different chromosomes in a genome. Like the stops that determine the major sound forms in an organ, the permanent connectrons (which are most probably driven by alarm signals from outside the cell) determine the major aspects of gene expression behavior. In the same way that certain patterns of stops will be used for toccatas and others for fugues, we can expect to find permanent connectrons associated with cell-cycle, change of energy sources, and even external calls for the cell to commit suicide (i.e. apoptosis). In the organ analogy the music (i.e. the notation component) is separate from the instrument itself. The organist can bring any piece of music to an instrument and play it. Genomes occasionally receive DNA from outside sources. Although it may be stretching the analogy a bit, one might argue that the cell might “play” the new DNA to see if it confers any new evolutionarily advantageous properties. In the basic method patent we showed that some connectrons are controlled by promoters that do not produce an Open Reading Frame (ORF). These ORF-less transcripts do include C1/C2 sequences. As we have processed more prokaryotic, archeal and eukaryotic genomes to determine their connectron structure, the number of short gene-like fragments called pseudo-genes have increased. In the eukaryotic genomes the size of the human genome (i.e. 3.5 billion bases), there is still about 90% of the genomic DNA that is not well characterized. It may be that this is where the “music” of the cell is stored. The utilization of this program may be able to resolve this question.

This invention is a program method for the simulation of cellular gene expression behavior by means of the interaction of permanent and transient connectrons along with the iRNAs and the stRNAs.

### PRIOR ART

The Prior Art disclosed in my above identified Patent Application is incorporated herein by reference.

### BRIEF DESCRIPTION OF THE OBJECTS OF THE INVENTION

The object of the invention is to provide a method for using permanent and transient connectrons and/or iRNAs and stRNAs to show how connectrons control the expression of the genes in a cell.

### DESCRIPTION OF THE DRAWINGS

The above and other objects, advantages and features of the invention will become more apparent when considered with the following specification and accompanying drawings wherein:

FIG. 1 illustrates that (a) Complex Representation of Connectron Formation. (b) Simplified Representation of Connectron Formation,

FIG. 2 illustrates that using the simplified notation (a) At first Genes c1, c2 and c3 are free to be expressed under ordinary promotional control. (b) Then Gene b2 begins to express thus forming a connectron that turns off the expression of Genes c1, c2 and c3,

FIG. 3 illustrates that (a) Gene a1 begins to express thus forming a connectron that turns off the expression of Genes b1, b2 and b3. (b) As a result, the connectron that turned off the expression of Genes c1, c2 and c3 is eliminated at the end of its lifetime and then Genes c1, c2 and c3 are capable once again of being expressed under promotional control,

FIG. 4 illustrates that (a) If Gene c2 happens to express, it will generate a connectron that controls the expression of Genes a1 and a2. (b) As a result, the connectron formed by Gene a1 is allowed to expire at the end of its lifetime thus making it possible for Genes b1, b2 and b3 to be expressed under ordinary promotional control,

FIG. 5 illustrates that if Gene d1 is only under promotional control, then it will generate a permanent connectron that controls the expression of Genes c1, c2 and c3. The permanent connectron generated by Gene d1 will break the cycle of gene expression control among the “a”, “b” and “c” genes,

FIG. 6 illustrates that (a) Gene a1 can exert control over Cycle 1 while Gene b1 can exert control over Cycles 2 and 3. (b) A portion of the C1/C2 of Gene b1 is different from the C1/C2 of Gene a1. When Gene b1 expresses, the iRNA suppresses the expression of Gene a1 thus modulating its control over Cycle 1,

FIG. 7 illustrates that (a) Two connectrons that are not in conflict. (b) Gene b1 cannot form a connectron. (c) Gene a1 can form a connectron because it includes the smaller Gene b1 connectron. This is the “Paper covers rock” rule,

FIG. 8 illustrates that (a) Gene b1 cannot form a connectron. (b) Gene b1 cannot form a connectron. (c) Gene b1 can form a connectron as long as the T2 sequence of the Gene a1 connectron is separated from the T1 sequence of the Gene b1 connectron,

FIGS. 9 to 13 details the structure of the computer program that simulates the interaction of connectrons and iRNA,

FIG. 14 is a simulation of E. coli using random initial conditions, and

FIG. 15 is a plot of the number of changes in connectron activity during a simulation of E. coli using random initial conditions.

### DESCRIPTION OF THE INVENTION

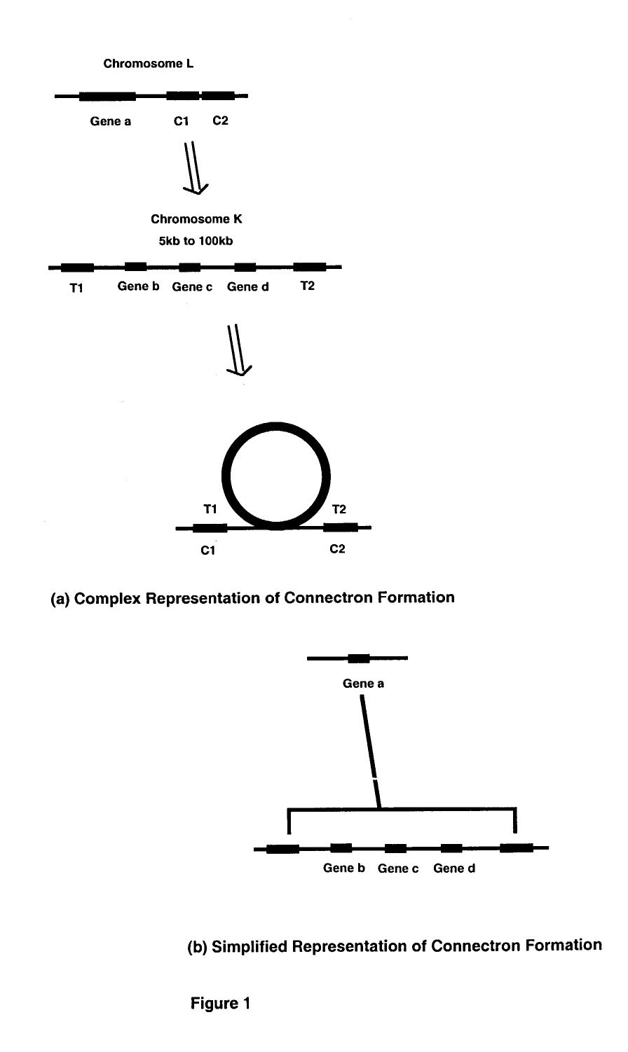

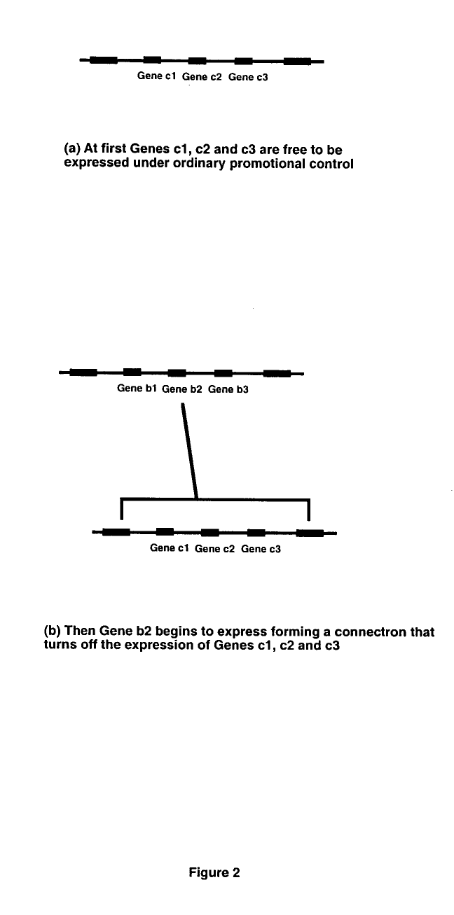

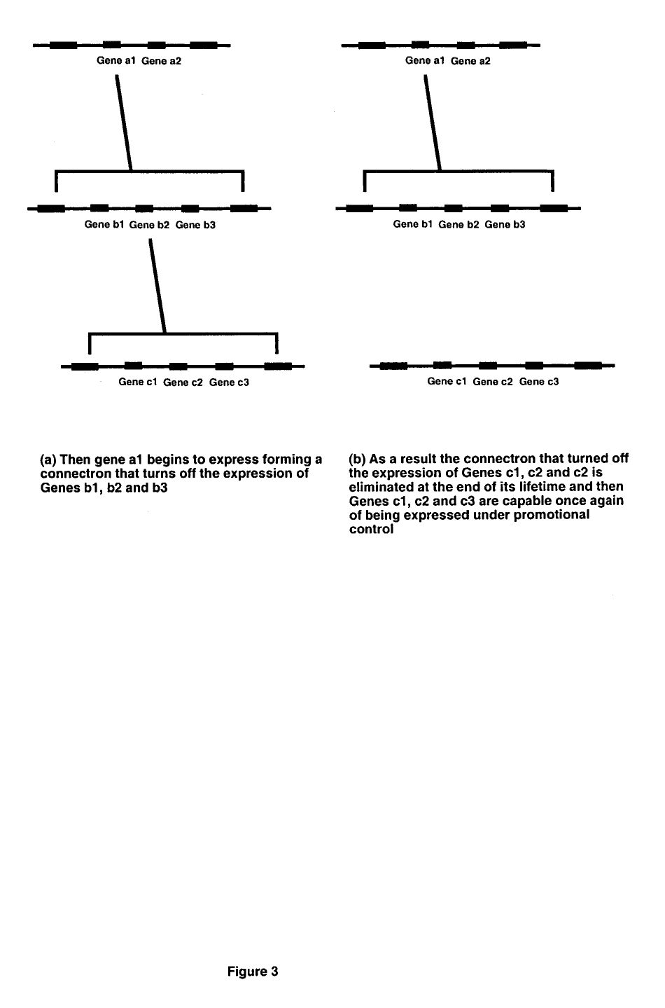

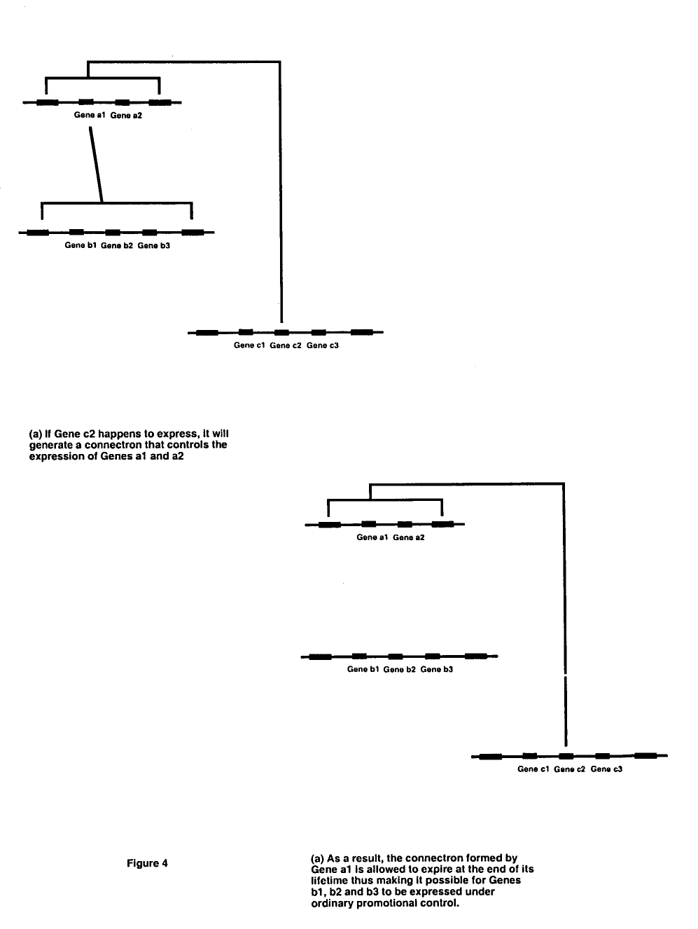

The interaction of the connectrons and the iRNAs and stRNAs in the genome of a cell generates a temporal dynamic. FIG. 1a shows the complex representation of the formation of a connectron. This representation names the chromosome on which the control gene and the C1/C2 sequences reside, as well as naming the chromosome on which the T1 and T2 sequences and the target genes reside. The simplified representation in FIG. 1b just shows that the control gene causes the formation of a connectron around the target genes. FIGS. 2, 3 and 4 describe the gene expression control behavior among three sets of genes—called a, b and c. In FIG. 2a, at first Genes c1, c2 and c3 are free to be expressed under ordinary promotional control. In FIG. 2b, Gene b2 begins to express thus forming a connectron that turns off the expression for Genes c1, c2 and c3. In FIG. 3a, Gene a1 begins to express thus forming a connectron that turns off the expression of Genes b1, b2 and b3. The result of this connectron formation is shown in FIG. 3b. As a result of the Gene b2 being turned off the connectron that turned off the expression of Genes c1, c2 and c3 is eliminated at the end of its lifetime because no more RNA is being generated by the expression of the Gene b2. When this connectron is allowed to expire, then Genes c1, c2 and c3 are capable of being expressed under ordinary promotional control. Now for the sake of this example, let us consider that the newly expressible Gene c2 forms a connectron that turns off the expression of the Genes a1 and a2. This action is shown in FIG. 4a. As a result of turning off the Genes a1 and a2, the connectron formed by Gene a1 that controls the expression of Genes b1, b2 and b3 is allowed to expire at the end of its lifetime. In this example we have a temporal cycle of gene expression control. A “b” gene turns off the “c” genes. An “a” gene turns off the expression of the “b” genes. A “c” gene turns off the expression of the “a” genes, etc. Once started, this cycle can continue indefinitely. If one of the controlling genes in this cycle is not expressed because of promotional control in the cellular environment, then the cycle of gene expression control will die away.

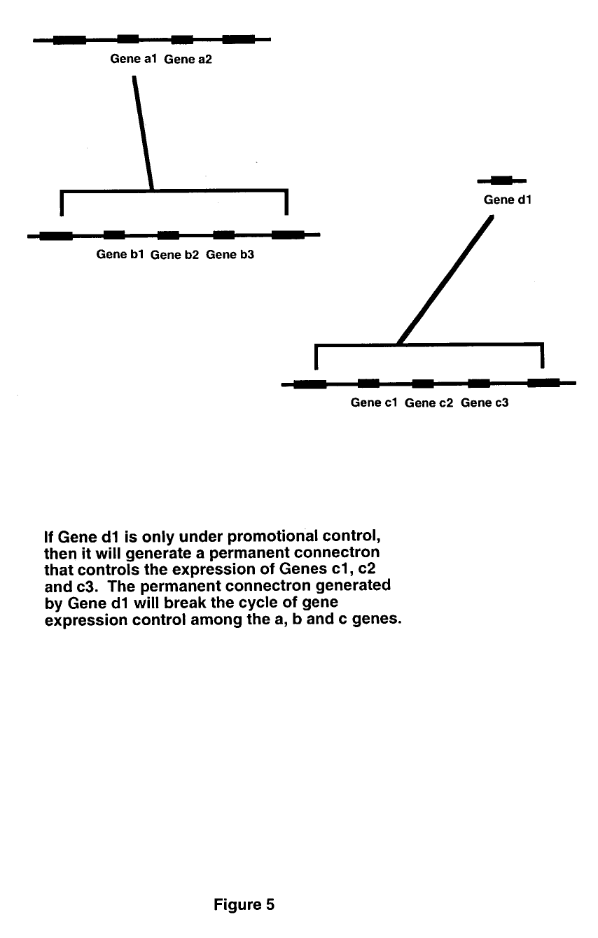

FIG. 5 shows how a permanent connectron can influence the behavior of the cycle shown in FIGS. 2 through 4. The expression of Gene d1 is only due to events in the cellular environment—not to any other connectron control. When Gene d1 expresses, it generates a connectron that turns of Genes c1, c2 and c3. With the “c” genes permanently turned off, they cannot be turned off by the expression of Gene b2. Likewise because the “c” genes are turned off permanently by Gene d1, the Gene c2 cannot turn off the “a” genes. In this example, the effect of the expression of the permanent connectron is to shut off the cycle of gene expression control among the “a”, “b” and “c” genes.

These examples are VERY simple. Real genomes are much, much more complex. Typical prokaryotic, Archeal and eukaryotic genomes have from 100 to 100,000 connectrons. The utility of the computer method described in this patent application is that it provides an experimental basis for investigating connectron-controlled behavior in naturally occurring and synthetic conditions.

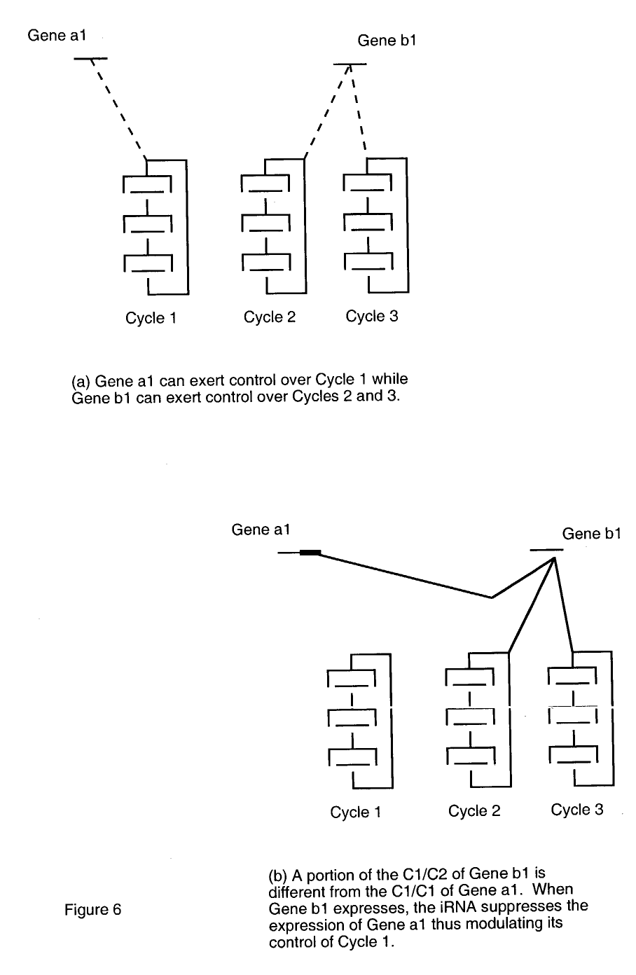

In FIG. 6 the cycle of gene expression control described in FIGS. 2 to 4 is further simplified. The numbers of genes within a connectron as well as their names have been eliminated. The three-stage cycle of temporal control is now just an abstract pattern. There could, of course, be more stages in the cycle. For the purpose of this example, in FIG. 6a Gene a1 can exert control over Cycle 1 and Gene b1 can exert control over Cycles 2 and 3. For the purpose of this example, let us assume that the C1/C2 of Gene b1 is contains a portion of the C1/C2 of Gene a1, but that Gene b1 also has a unique portion of its C1/C2 that controls Cycles 2 and 3. If Gene a1 expresses first then it just exerts control over Cycle 1 but if Gene b1 expresses first then it exerts control over cycles 2 and 3. In addition because there is common C1/C2 sequence between Genes b1 and a1 then the iRNA of Gene b1 will block the expression of the C1/C2 of Gene a1. In this way Gene b1 can block the control of Cycle 1 by Gene a1. This is a typical way in which iRNA and stRNAs exert control over cellular behavior.

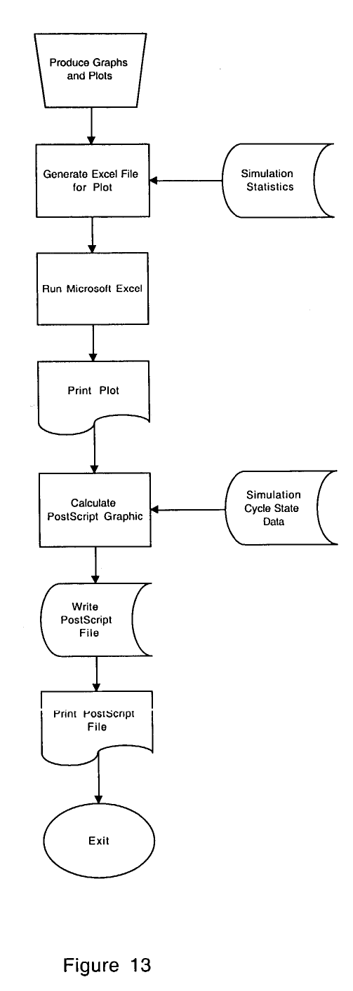

The interactions of the connectrons in a genome form an abstract state machine. The state of the machine is determined by the pattern of gene groups that are turned off. An important component of this program invention is the development of a graphic capable of representing the complexity of each state as well as presenting a large number of states for visual examination. In FIG. 13 such a graphic is presented.

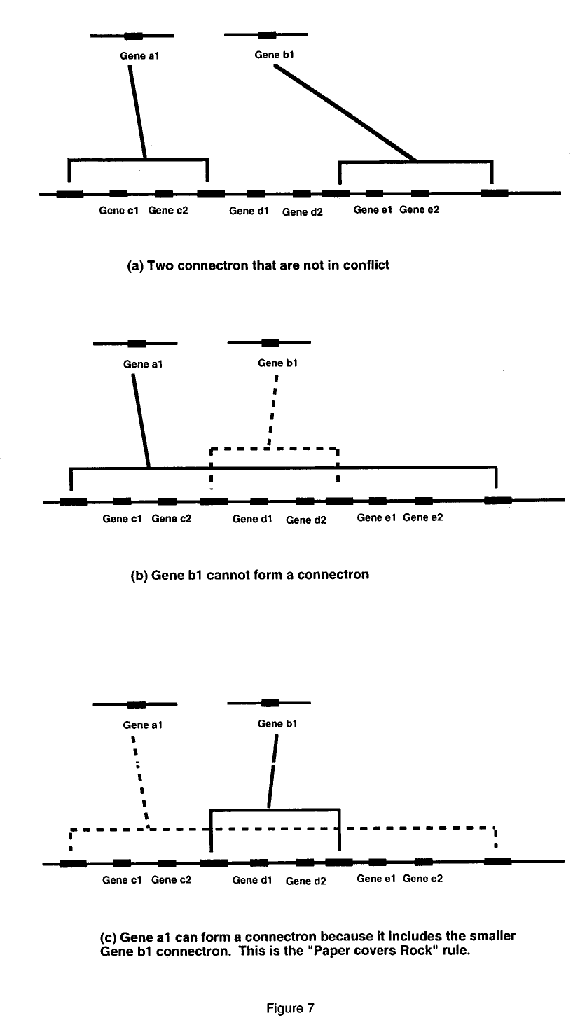

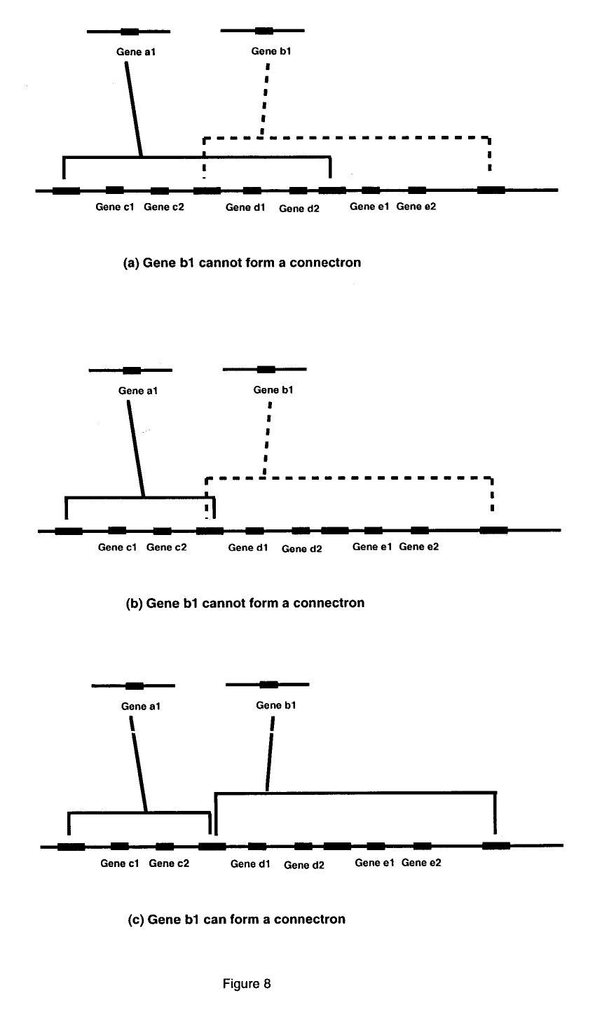

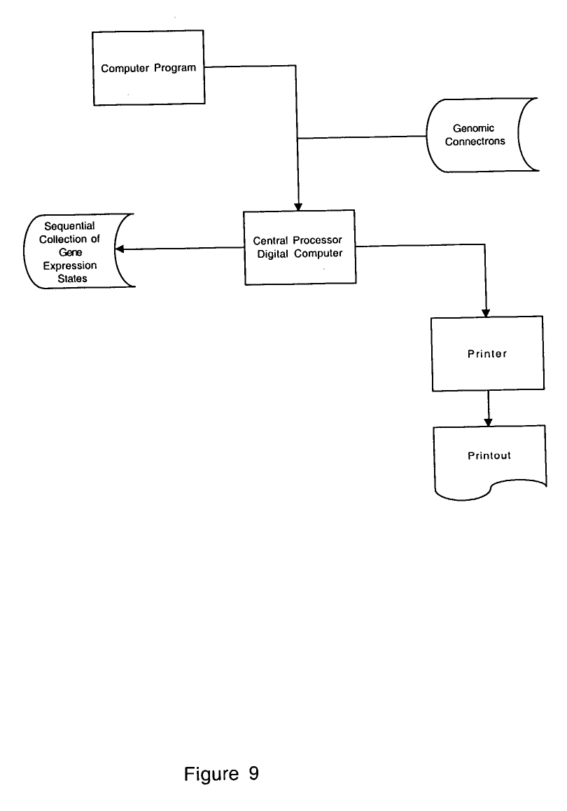

The key element in the computer program shown in FIGS. 9 to 12 is the set of rules for how connectrons interact. FIG. 7a shows that two connectrons that do not share any sequence elements can both form. This is particularly true if the two connectrons are on different chromosomes. In FIG. 7b the connectron generated by Gene a1 forms first. When Gene b1 expresses, its C1/C2 RNA cannot form a connectron because the corresponding T1-T2 is inaccessible. FIG. 7c shows that although Gene b1 has formed a connectron, the connectron produced by the expression of Gene a1 can also form. There is a children's game called “Paper, Scissor, Rock”. In this game “Paper covers Rock”, “Scissor cuts Paper” and “Rock breaks Scissor”. The application of this rule may be subjective but the physical implementation in DNA is plausible. Further computational experimentation may resolve the utility of the “Paper covers Rock” rule. FIG. 8a shows that Gene a1 has formed a connectron first. Therefore the later expression of Gene b1 cannot form a connectron. FIG. 8b shows that the expression of Gene a1 has formed a connectron. The C1 produced by the expression of Gene b1 tries to use a portion of the T2 of the Gene a1 connectron. This type of connectron does not have a plausible physical implementation. In FIG. 8c the two connectrons share a common T2-T1 sequence. In this case the two connectrons can form because there is a plausible physical implementation—although only just.

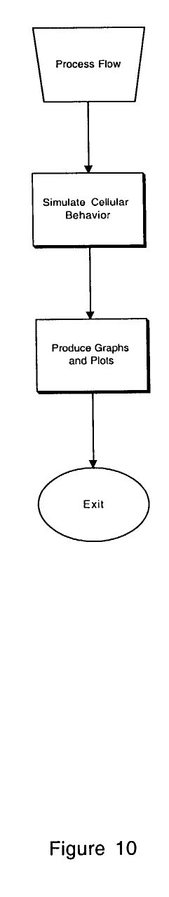

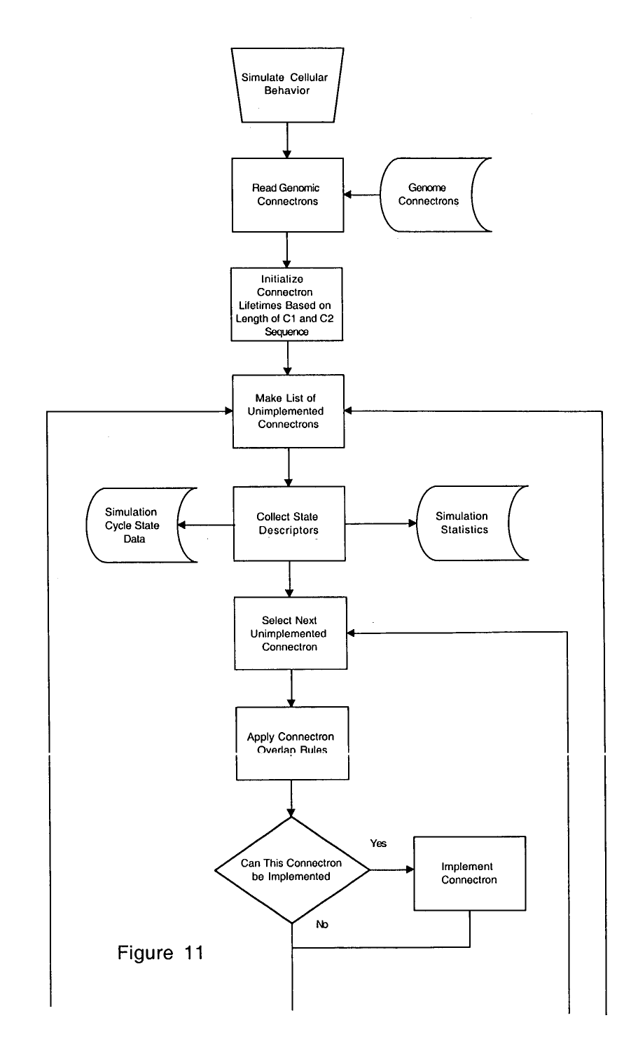

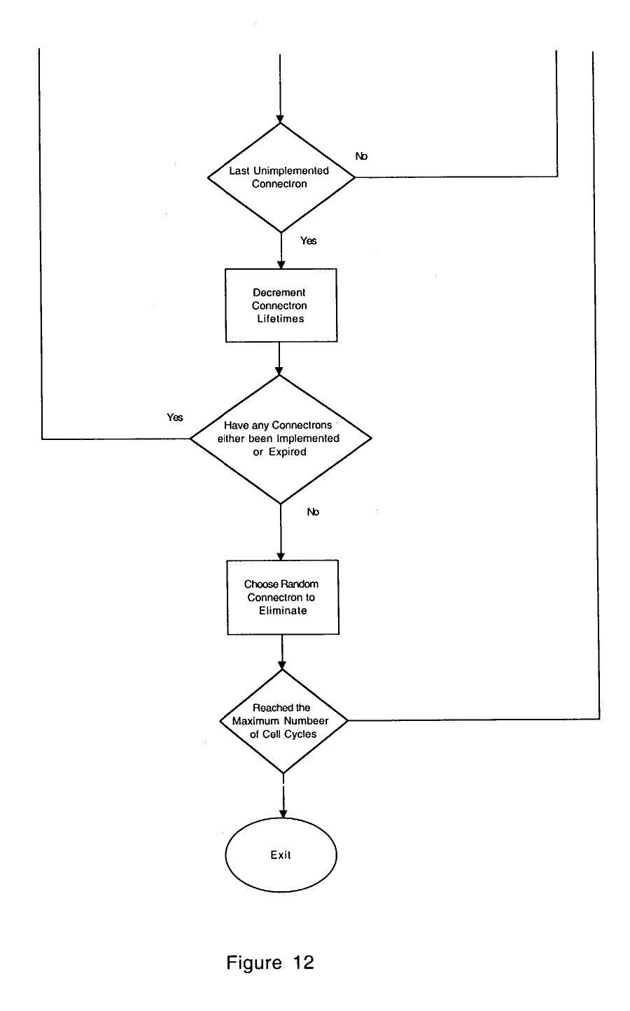

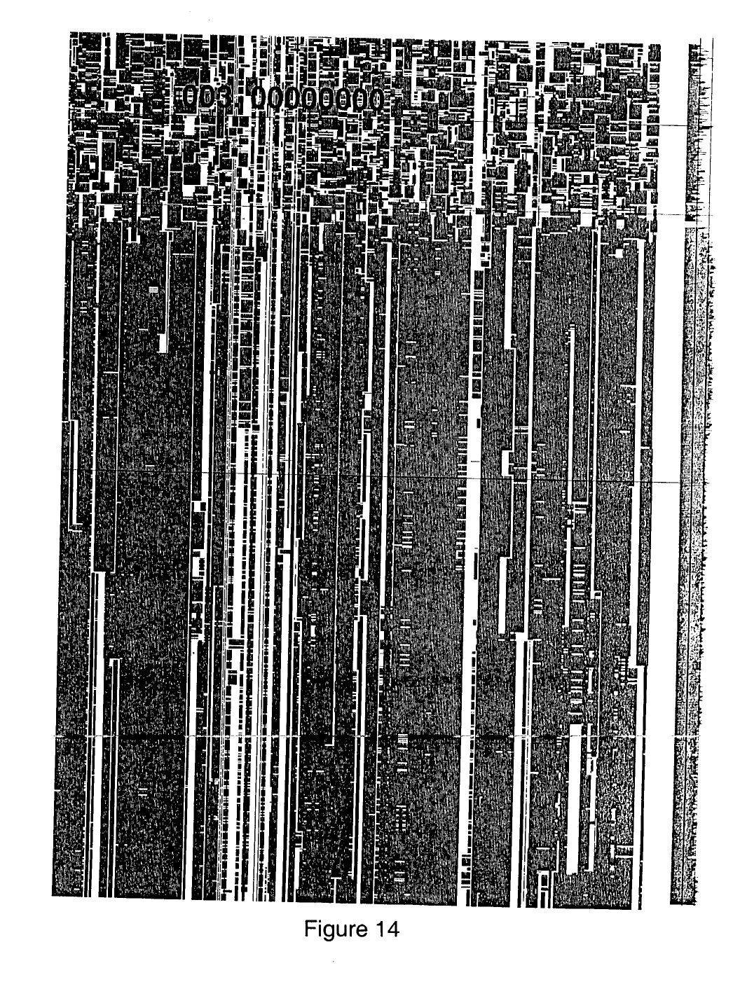

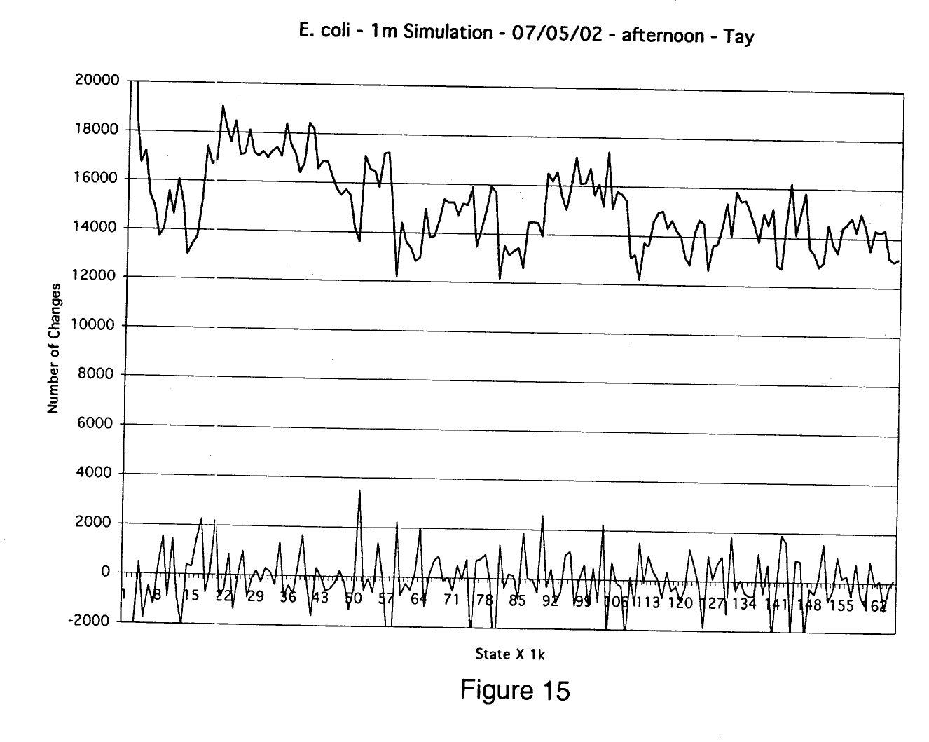

FIGS. 9 through 13 detail the structure of the program that simulates the interaction of connectrons. FIG. 9 is the general structure of the computer, the program, the data files and the printing operation. FIG. 10 shows the process flow of the program. FIGS. 11 and 12 describe the dominant calculation process in the program. In conjunction with the connectron conflict resolution rules described above, this process does the basic simulation of connectron and iRNA interaction. Along with knowledge of our basic methods patent application, someone skilled in the art should be able to take this diagram and reproduce the cell simulation behavior. FIG. 13 describes the peripheral processes for generating, printing and plotting the cell simulation data that are shown in FIGS. 14 and 15.

FIG. 14 shows a simulation of the E. coli genome. Each vertical line is one group of genes. The presence of a vertical line indicates that the group of genes is turned off by some connectron. The horizontal lines at the right of the figure show the percentage of the gene groups turned off. The lower limit (i.e. the leftmost edge) of this graph is 50% of the gene groups turned off. The two other vertical lines are 60% and 70% of the gene groups turned off. Running down the page, this side-graph shows that as the simulation proceeds, between 75% and 85% of the gene groups are turned off. The vertical stripes on the left side of this graph show that the gene groups that are turned off change quite rapidly and dramatically. There are 1,000 simulation states down the whole page. For the first 100 simulation states the lifetimes of the connectrons are randomized and kept small. This corresponds to a heating phase. From simulation states 101 to 200 the lifetimes of the connectrons are increased from zero to a value determined by the length of the shortest match between the (C1 and T1) sequences and the (C2 and T2) sequences. From simulation state 201 to 1,000 the simulation runs in it normal mode. The simulation produces extraordinarily complex behavior. Part of the utility of this invention is that it will enable us to study small and large, as well as simple and complex genomic systems (i.e. cells). By varying the lifetimes of the connectrons as well as the iRNA and stRNAs, it will be possible to produce a large variety of behaviors.

FIG. 15 Shows the results of doing a larger scale simulation. The upper curve is the number of connectrons going into and out of existence during a 1,000 state period. The lower curve is rate of change in a 1,000 state period. The cellular simulation program described in this invention is relatively inexpensive to run in terms of computer time. As a basic cellular simulation tool, this invention will become a workhorse for computational experimentation. The rules of interaction between the connectrons, as well as the time constants associated with the various connectrons and iRNAs can be easily changed.

This invention utilizes the capabilities in application Ser. No. ______ filed contemporaneously herewith and entitled “Determination of interference RNAs (iRNAs) and small temporal RNAs (stRNAs) and their interaction with connectrons in prokaryotic, archea and eukaryotic genomes”. The iRNAs and stRNAs play a vital role in determining the simulation of cellular dynamics.

This invention shows that the ideas of connectrons and interference RNA are very powerful. The computation process described in our basic methods patent application generates for a given genomes a number of connectrons. At first one might assume that these connectrons are static entities. This invention demonstrates that connectrons and iRNA do indeed interact with each other in a parallel yet sequential manner. If a connectron once formed stayed in existence forever, then there would be no temporal dynamic. It is precisely because the connectrons and the iRNA constructs have (triple-stranded generalized Hoogsteen helix determined) lifetimes that the whole genome can exhibit responsive and regulatory behavior. Nature seems to have used a large number of very simple relationships (i.e. the expression of one gene turns off the expression of other genes) to produce very complex behavior. FIG. 15 shows that the behavior of the E. coli genome is indeed very complex. The utility of this invention will hopefully be that many scientists throughout the world can use this tool to understand and explore the regulatory behavior of many different genomes ranging from the simplest bacteria through the ubiquitous (in the sea) Archea to the plants, animals and mammals that form our global biology.

What is claimed is:

1. A method for using permanent and transient connectrons and/or iRNAs and stRNAs to control the expression of the genes in a cell comprising determining, by computer, the interaction of said permanent and transient connectrons and/or iRNAs and stRNAs.

2. A method for using permanent and transient connectrons and/or iRNAs and stRNAs to elucidate the control of the expression of the genes in a cell comprising determining, by computer, the interaction of said permanent and transient connectrons and/or iRNAs and stRNAs.
